(19) United States
(12) Patent Application Publication
(10) Pub. No.: US 2014/0288179 A1
(43) Pub. Date: Sep. 25, 2014

(54) STABLE SOLUTIONS OF TREPROSTINIL

(71) Applicant: United Therapeutics Corporation, Silver Spring, MD (US)

(72) Inventors: David Zaccardelli, Cary, NC (US); Roger Andrew Jeffs, Chapel Hill, NC (US)

(21) Appl. No.: 14/302,816

(22) Filed: Jun. 12, 2014

Related U.S. Application Data
(63) Continuation-in-part of application No. 14/158,091, filed on Jan. 17, 2014.
Application No. 14/158,091 is a continuation of application No. 13/912,753, filed on Jun. 7, 2013, now Pat. No. 8,653,137.
Application No. 13/912,753 is a continuation of application No. 13/022,005, filed on Feb. 7, 2011, now Pat. No. 8,658,694.
Application No. 13/022,005 is a continuation of application No. 12/276,707, filed on Nov. 24, 2008, now Pat. No. 7,999,007.
Application No. 12/276,707 is a continuation of application No. 12/205,200, filed on Sep. 5, 2008, now abandoned.
(60) Provisional application No. 60/970,716, filed on Sep. 7, 2007.

(51) Int. Cl. A61K 47/18; A61K 31/557

(52) U.S. Cl. CPC A61K 47/183; A61K 31/557
USPC 514/569

(57) ABSTRACT

An improved method for administering treprostinil or a salt is described that allows for use of the diluted solution prior to administration for a longer period of time to avoid wasted medication.

### CROSS REFERENCE TO RELATED APPLICATIONS

This application is a Continuation-in-part of U.S. patent application Ser. No. 14/158,091, filed Jan. 17, 2014, which is a Continuation of U.S. patent application Ser. No. 13/912,753, filed Jun. 7, 2013, which is a Continuation of U.S. patent application Ser. No. 13/022,005, filed Feb. 7, 2011, which is a Continuation of U.S. patent application Ser. No. 12/276,707, filed Nov. 24, 2008, now U.S. Pat. No. 7,999,007, which is a Continuation of U.S. patent application Ser. No. 12/205,200, filed Sep. 5, 2008, which claims priority to U.S. Patent Application 60/970,716, filed Sep. 7, 2008, the entire contents of which are incorporated herein by reference.

### FIELD OF THE INVENTION

The present invention relates generally to the field of buffer solutions having bacteriostatic and/or bactericidal activity. More specifically, the present invention relates to buffer solutions that have bactericidal activity preferentially against gram negative bacteria.

### BACKGROUND OF THE INVENTION

The use of buffers to maintain a pH and solubilize or dilute active pharmaceutical agents (“APIs”) before administration (e.g., by injection) is routine. Many buffers, however, contain components that maintain a neutral pH and foster microbial growth, which can lead to sepsis and other undesirable infection-related complications.

Gram negative bacteria are a particularly troublesome class of microbes, as they are commonplace in the hospital environments and difficult to eradicate and/or control. Infections with this class of bacteria tend to have higher morbidity/mortality rates when a patient becomes septic, in part, because gram negative bacteria are especially difficult organisms to treat. Also, gram negative bacteria are associated with water contamination which can occur with chronic indwelling catheters such as used with intravenous administration. Hence, there is a need for buffer systems that have anticidal activity with specificity to gram negative bacteria.

REMODULIN® is an FDA approved product for treating pulmonary hypertension, which uses a solution of treprostinil that may be diluted before administration to a patient with a diluent having a pH of greater than about 10 (known as “Sterile Diluent for Flolan™” or “Sterile Diluent for Epoprostenol Sodium”). Other diluents included in the FDA approved labeling include sterile water for injection and 0.9% sodium chloride injection. However, the approved FDA label for REMODULIN® states that the solution of treprostinil is stable after being diluted at room temperature for only up to 48 hours. Accordingly, based on the current use guidance, medication will be wasted if the diluted solution is not used within 48 hours.

### SUMMARY OF THE INVENTION

In one embodiment of the present invention, a method of selectively killing gram negative bacteria and inhibiting the growth of gram positive bacteria in a pharmaceutical preparation comprising an active agent is provided, the method comprising supplying the active agent with a buffer having a pH of greater than about 10 or less than about 4.5 and a low buffer capacity, wherein the pharmaceutical preparation does not comprise epoprostenol sodium as the sole active agent. In addition to bacteria, the buffer may further inhibit the growth of fungus, mold, or both. Preferably, the buffer has a pH between about 10 to about 12, more preferably a pH between about 10.2 to about 10.8. In other embodiments, the buffer has a pH between about 3 and 4.5, more preferably a pH between about 3.5 and 4.5.

The buffer may comprise glycine; and in a specific embodiment, the buffer is sterile diluent for FLOLAN®, namely a buffer comprising glycine and sodium hydroxide, added to adjust the pH to 10.2 to 10.8. The active agent may be any active pharmaceutical agent that requires solution or dilution with a buffer and may be injected (e.g., intravenously). The active agent may be treprostinil sodium (sometimes referred to herein as treprostinil), preferably supplied at a concentration between about 0.004 mg/mL to about 0.13 mg/mL treprostinil sodium.

The buffer may comprise sorbic acid or citric acid or any other weak acid that is pharmaceutically acceptable for parenteral use. The pH can be adjusted with hydrochloric acid or sodium hydroxide to attain a final pH between 3 and 4.5. The active agent may be any active pharmaceutical agent that requires solution or dilution with a buffer and may be injected (e.g., intravenously).

In another embodiment of the invention, a method of reducing the occurrence of blood stream infections in a mammal being treated with an active agent is provided, the method comprising administering to the mammal the active agent with a buffer having a pH of greater than about 10 or less than about 4.5 and a low buffer capacity, wherein the active agent is not epoprostenol sodium, and wherein the administration reduces the gram negative bacteria and inhibits the growth of gram positive bacteria. In some cases, the human subject may suffer from pulmonary arterial hypertension.

Preferably, the buffer has a pH between about 10 to about 12, more preferably a pH between about 10.2 to about 10.8 and a low buffer capacity. Alternatively, the buffer has a pH preferably between about 3 to about 4.5, more preferably a pH between about 3.5 to about 4.5 and a low buffer capacity. The buffer may comprise glycine; and in a specific embodiment, the buffer is sterile diluent for FLOLAN®. The active agent may be any active pharmaceutical agent that requires solution or dilution with a buffer and may be injected (e.g., intravenously). The active agent may be treprostinil sodium, preferably supplied at a concentration between about 0.004 mg/mL to about 0.13 mg/mL treprostinil. Selection of the buffer will depend on the desired pH. While the buffer components should have a pKa close to the desired pH, the buffer capacity should be low to avoid pH changes in the blood upon infusion. A preferred buffer capacity for such buffers is 0.01 and less.

One embodiment of the present invention relates to a method of treating pulmonary hypertension, comprising, prior to administration to a patient, combining treprostinil or a pharmaceutically acceptable salt thereof with a diluent providing a pH of greater than about 10 to form a diluted solution of treprostinil, starting to administer the diluted solution of treprostinil to a patient after a period greater than 48 hours following dilution, preferably after a period of time following dilution of up to 3, 4, 5, 6, 7, 8, 9, 10, 11, 12, 13, 14, 15, 16, 17, 18, or 19 days. Preferably, the diluent has a pH between about 10 to about 12, more preferably a pH between about 10.2 to about 10.8.

The diluent may optionally further comprise glycine or arginine. In a specific embodiment, the diluent is sterile diluent for Flolan™, which is supplied in vials containing 50 mL of 94 mg glycine, 73.3 mg sodium chloride, sodium hydroxide (added to adjust pH), and Water for Injection, USP.

The treprostinil to be diluted may optionally be supplied in a 20 mL vial containing 20, 50, 100, or 200 mg of treprostinil at a concentration of 1 mg/mL, 2.5 mg/mL, 5 mg/mL or 10 mg/mL. An intravenous infusion system reservoir, such as a cassette, optionally having a volume of 50 or 100 mL is used to contain the diluted solution of treprostinil. In other embodiments, instead of an intravenous infusion system, an implantable pump is provided for delivery of the diluted solution of treprostinil.

### BRIEF DESCRIPTION OF THE DRAWINGS

FIGS. 1A and 1B are chromatograms of a “blank” injection of BWFI (A) and treprostinil diluted with BWFI (B).

FIGS. 2A and 2B are chromatograms of a “blank” injection of BNS (A) and treprostinil diluted with BNS (B).

FIGS. 3A and 3B are chromatograms of Sterile Diluent for FLOLAN® (A) and treprostinil diluted with same (B).

FIGS. 4A and 4B are chromatograms of 0.004 mg/mL treprostinil in Sterile Diluent for FLOLAN® at T₀ (A) and Tᵢₙᵢₜᵢₐₗ (B).

FIGS. 5A and 5B are chromatograms of 0.13 mg/mL treprostinil in Sterile Diluent for FLOLAN® at T₀ (A) and Tᵢₙᵢₜᵢₐₗ (B).

FIG. 6 is a graph showing the antimicrobial activity (CFU) over time (days) of various buffer systems against Staphylococcus aureus, a gram positive bacterium, in a pharmaceutical preparation comprising 0.004 mg/mL. Values for ≦Log 1 (treprostinil in sterile diluent) or ≧Log 6.48 (treprostinil in WFI, NS) are recorded as Log 1 and Log 6.48, respectively.

The legend for FIGS. 6-15 is as follows:
(open circles): FLOLAN® in Sterile Diluent for FLOLAN®
(closed circles): Treprostinil in Sterile Diluent for FLOLAN®
(open squares): Treprostinil in sterile water for injection
(closed squares): Treprostinil in bacteriostatic water for injection
(open diamonds): Treprostinil in normal (0.9%) saline
(closed diamonds): Treprostinil in bacteriostatic normal saline
(open triangle): Treprostinil in 5% dextrose in water for injection (D5W).

FIG. 7 is a graph showing the antimicrobial activity (CFU) over time (days) of various buffer systems against Escherichia coli, a gram negative bacterium, in a pharmaceutical preparation comprising 0.004 mg/mL treprostinil. Values for ≦Log 1 (treprostinil in sterile diluent) recorded as Log 1.

FIG. 8 is a graph showing the antimicrobial activity (CFU) over time (days) of various buffer systems against Pseudomonas aeruginosa, a gram negative bacterium, in a pharmaceutical preparation comprising 0.004 mg/mL treprostinil. Values ≦Log 1 (treprostinil in sterile diluent) or ≧6.48 (treprostinil in D5W) are recorded as Log 1 and Log 6.48, respectively

FIG. 9 is a graph showing the antimicrobial activity (CFU) over time (days) of various buffer systems against Candida albicans, a fungus, in a pharmaceutical preparation comprising 0.004 mg/mL treprostinil.

FIG. 10 is a graph showing the antimicrobial activity (CFU) over time (days) of various buffer systems against Aspergillus niger, a mold, in a pharmaceutical preparation comprising 0.004 mg/mL treprostinil.

FIG. 11 is a graph showing the antimicrobial activity (CFU) over time (days) of various buffer systems against Staphylococcus aureus, a gram positive bacterium, in a pharmaceutical preparation comprising 0.13 mg/mL treprostinil. Values for ≦Log 1 (treprostinil in sterile diluent) or ≧Log 6.48 (treprostinil in WFI, NS, D5W) are recorded as Log 1 and Log 6.48, respectively.

FIG. 12 is a graph showing the antimicrobial activity (CFU) over time (days) of various buffer systems against Escherichia coli, a gram negative bacterium, in a pharmaceutical preparation comprising 0.13 mg/mL treprostinil. Values for ≦Log 1 (treprostinil in sterile diluent) or ≧Log 6.48 (treprostinil in NS) are recorded as Log 1 and Log 6.48, respectively.

FIG. 13 is a graph showing the antimicrobial activity (CFU) over time (days) of various buffer systems against Pseudomonas aeruginosa, a gram negative bacterium, in a pharmaceutical preparation comprising 0.13 mg/mL treprostinil. Values for ≦Log 1 (treprostinil in sterile diluent) recorded as Log 1.

FIG. 14 is a graph showing the antimicrobial activity (CFU) over time (days) of various buffer systems against Candida albicans, a fungus, in a pharmaceutical preparation comprising 0.13 mg/mL treprostinil. Value≧Log 3.48 at time 0.25 hours for treprostinil in NS recorded as Log 3.48.

FIG. 15 is a graph showing the antimicrobial activity (CFU) over time (days) of various buffer systems against Aspergillus niger, a mold, in a pharmaceutical preparation comprising 0.13 mg/mL treprostinil.

### DETAILED DESCRIPTION OF THE PREFERRED EMBODIMENTS

The present invention is directed to the use of buffer systems to maintain a specific pH range as anticidal agents in pharmaceutical preparations. The term “buffer” as used herein refers to any solution with a controlled pH that may serve to dissolve a solid (e.g., lyophilized) pharmaceutical or as a diluent to dilute a liquid pharmaceutical. According to the invention, the buffers described herein maintain a pH that exhibits bacteriostatic activity toward most, if not all, microbes, including bacteria, molds and fungi and further exhibit bactericidal activity toward gram negative bacteria. Examples of gram negative bacteria include Escherichia coli, Pseudomonas aeruginosa, Salmonella, Moraxella, Helicobacter, Stenotrophomonas, Bdellovibrio, Legionella, Neisseria gonorrhoeae, and Neisseria meningitidis. Gram negative bacteria are a common source of infection in hospital environments and therefore buffers that maintain a pH above 10 or less than about 4.5 with low buffer capacity have bactericidal activity specific for gram negative bacteria are desirable. By way of example, gram positive bacteria include Staphylococcus aureus Bacillus, Listeria, Staphylococcus, Streptococcus, Enterococcus, and Clostridium.

“Bacteriostatic” is defined as the ability to retard or prevent the expansion of a microbe that might be present, or become present, in the buffer solution. In other words, “bacteriostatic” activity does not include bactericidal activity, which is defined herein as activity that kills a microbe that might be present, or become present, in the buffer. Microbes are broadly defined herein to include unicellular organisms, such as, for example, bacteria, molds, and fungi.

The present inventors have learned that buffers having high pH (>10) or low pH (<4.5) have bactericidal activity specific for gram negative bacteria and bacteriostatic activity toward gram positive bacteria and other microbes. Without being held to or bound by theory, it is currently believed that differences in the biochemistry, perhaps cell wall biochemistry, between gram negative and gram positive bacteria may account for their differential sensitivity toward high pH buffers. In the context of the present invention, “high” pH is a pH value of about 9 to about 12, preferably about 10 to about 12. In a preferred embodiment of the invention, buffers have a pH of about 10.2 to about 10.8 or about 3.5 to about 4.5.

In addition to high pH, the present inventors have learned the buffers comprising glycine are particularly advantageous. In such embodiments, glycine is present at a concentration (w/w) of about 30% to about 80%, preferably about 45% to about 65%, and most preferably, about 50% to about 60%. The term “about” is used herein in recognition of the inherent inaccuracies in calculations and measurements in the art and to include nominal and accepted variations “about” the recited numeral.

In addition to glycine, buffers as described herein may comprise any other buffer system, including those known in the art, that can maintain a pH in the ranges stated herein.

In a specific embodiment of the present invention, the diluent for FLOLAN® (epoprostenol sodium) employs glycine as a buffer component. As will be described in greater detail below, the diluent for FLOLAN® was unexpectedly discovered to have specific anticidal activity toward gram negative bacteria and bacteriostatic activity toward remainder microbes. The diluent for FLOLAN® comprises 50 mL of 94 mg glycine, 73.3 mg sodium chloride, and sodium hydroxide, added to adjust the pH to 10.2 to 10.8. (About 44% NaCl in glycine.)

The buffers as described herein may be suitable for any active pharmaceutical ingredient (“API”) that is stable at high pH and provided that the chemical properties of the API do not substantially drop the pH of the buffer below, for example, about 10. Hence, the following examples notwithstanding, the present invention should not be limited to any one or any one class of API nor, for that matter, a limited range of concentrations. Further, the novel and unexpected anticidal properties of the buffers may be especially suited for medicaments that are administered by injection. Indeed, in one embodiment of the invention, it is anticipated that use of the high and low pH buffers as described herein can reduce the occurrence of blood stream infections in a mammal being treated with an active agent. It should be noted, however, that the present invention is not limited to medicaments that are prescribed for injection (including intravenous injection), but any medicament that requires solution and/or dilution (e.g., for oral administration).

In a specific embodiment of the present invention, the buffer systems described are used with treprostinil sodium. More specifically, as will be next disclosed by way of examples, the diluent for FLOLAN® is used to buffer treprostinil sodium.

### EXAMPLES

A compatibility study of treprostinil with a 100-mL CADD delivery device was performed. More specifically, the compatibility and stability of treprostinil diluted with bacteriostatic water for injection (“BWFI”) or bacteriostatic normal saline (“BNS”), both of which are preserved with parabens, was determined. The sample solutions were prepared at 0.004 mg/mL and 0.13 mg/mL treprostinil, which comprises the entire range of concentrations at which treprostinil might be prescribed, and placed in a SIMS Deltec, Inc. CADD-Legacy™ 1 (Model 6400) Pump delivery device that was pumped continuously over a period of 52 hours while stored at 40° C. and ambient relative humidity (“RH”).

At specified time points (e.g., T₀, initial, 24 hours, and 52 hours), samples were collected from the distal end of the tube after pump and characterized for appearance, pH, and concentration of treprostinil. Furthermore, the solutions were subjected to antimicrobial effectiveness testing (“AET”) over a similar time period of about 2 days. A similar experimental procedure was followed for Flolan reconstituted solutions. However, sterility and AET testing were performed on FLOLAN® after only 8 hours at room temperature on account of the medicament's limited stability in solution.

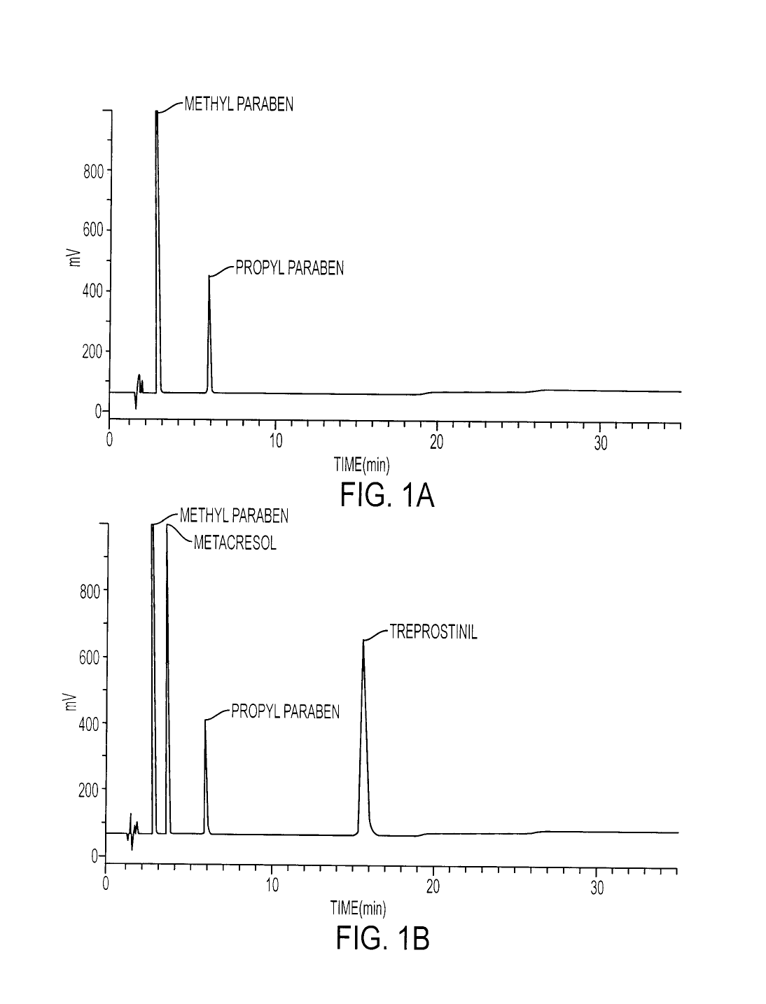

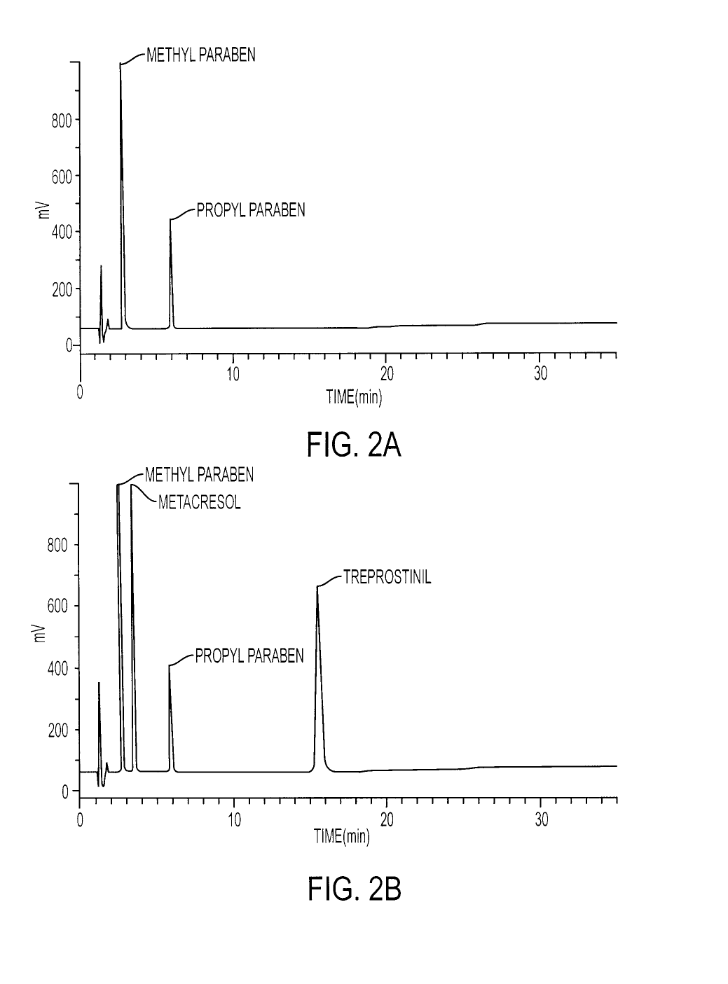

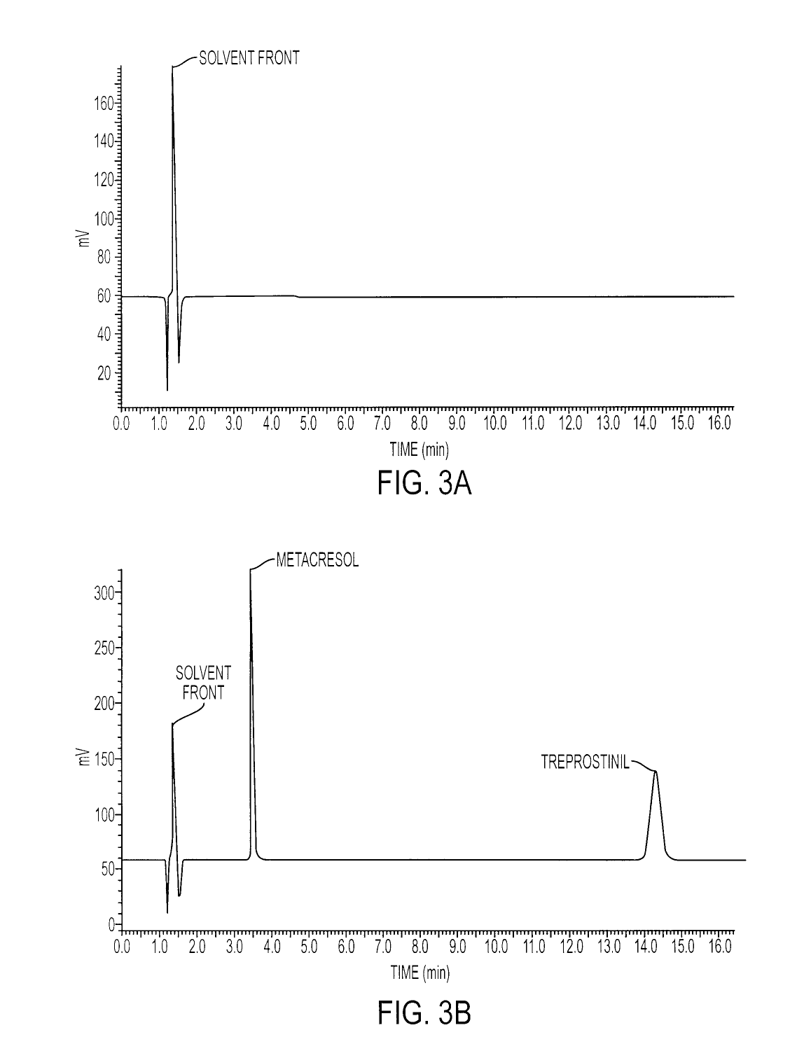

The stability of treprostinil was monitored by a fully validated stability indicating HPLC assay. In order to ascertain whether parabens—present in the “bacteriostatic” solutions—would cause interference in the chromatography with treprostinil, a preliminary experiment confirmed that the paraben “peaks” did not interfere with the treprostinil “peak” Or any impurity “peak.” Solutions of BWFI and BNS, and treprostinil diluted in BWFI and BNS were analyzed using HPLC. FIGS. 1 and 2 shows that the paraben peaks from either methyl- or ethyl-paraben did not interfere (e.g., overlap) with the peak for treprostinil. There was also no chromatographic interference of treprostinil with Sterile Diluent for FLOLAN® (FIG. 3).

A low-level linearity study was also performed to cover the expected concentration range of treprostinil in the dilute solutions. Five solutions of treprostinil were prepared at 0.002, 0.01, 0.05, 0.1, and 0.15 mg/mL (diluted from the 1.0 mg/mL standard solution) and each solution was injected in duplicate. The intention was to prove linearity between the detector response and treprostinil concentration within the diluted concentration range in order to use a single-point standard at 0.1 mg/mL during the analysis. The detector response for treprostinil was determined to be linear from 0.002 to 0.15 mg/mL. The correlation coefficient (r) for the experiment was 0.999995, meeting the requirement of at least 0.999.

Solutions of 0.004 mg/mL treprostinil in BWFI, BNS, or Sterile Diluent for FLOLAN® were prepared from the 1.0 mg/mL strength of REMODULIN®. Solutions of 0.13 mg/mL treprostinil in BWFI and in BNS were prepared from the 10 mg/mL strength of REMODULIN®. Vials of FLOLAN® were reconstituted with 5 mL of Sterile Diluent for FLOLAN® using the procedure outlined in the package insert.

A portion (approximately 2 mL) of each of the four solutions was removed for T₀ analysis. The remaining solution was loaded into each of four separate SIMS Deltec, Inc. 100-mL Medication Cassette™ Reservoirs. The cassettes and tubing were attached to the CADD-Legacy™ 1 Pump following the manufacturers instructions. The four cassette/CADD pump sets were placed in a 40° C./Ambient RH chamber. A needle at the end of the tubing was placed into a sealed HPLC vial (with needle vent). The flow on the pump was set to 40 mL/24 hours and started. The solution from each pump was collected into separate HPLC vials (for about an hour) for testing at the “Initial” interval. The needle was then transferred to a sealed waste container (with needle vent). At 24 and 52 hours, the solution was collected again into a new, sealed HPLC vial for testing.

The solutions collected at T₀, initial, 24 hours, and 52 hours were analyzed for physical appearance, pH, and assayed by HPLC for treprostinil. Tables 1 and 2 summarize the results for treprostinil diluted with BWFI and BNS, respectively. The appearance of all solutions was clear and colorless, free from visible particulate matter. Hence, the results show no compatibility problems for the treprostinil solution in BWFI or BNS at either concentration.

TABLE 1
Chemical testing results for treprostinil in BWFI

| | | | Testing Interval | | | |
|---|---|---|---|---|---|---|
| Concentration | | Prep- | | | 24 | 52 |
| (mg/mL) | Testing | aration | T₀ | Tᵢₙᵢₜᵢₜₐₗ | hours | hours |
| 0.004 | Treprostinil | 1 | 104.1 | 99.5 | 100.8 | 99.6 |
| | Assay | 2 | 100.6 | 100.6 | 101.1 | 101.0 |
| | (% LC) | Average | 102.4 | 100.0 | 101.0 | 100.3 |
| | pH | NA | 6.7 | 6.8 | 6.8 | 6.8 |
| 0.13 | Treprostinil | 1 | 100.1 | 99.6 | 100.4 | 101.2 |
| | Assay | 2 | 100.0 | 99.8 | 100.5 | 100.9 |
| | (% LC) | Average | 100.0 | 99.7 | 100.5 | 101.0 |
| | pH | NA | 6.7 | 6.9 | 6.8 | 7.0 |

LC: Label claim

TABLE 2
Chemical testing results for treprostinil in BNS

| | | | Testing Interval | | | |
|---|---|---|---|---|---|---|
| Concentration | | Prep- | | | 24 | 52 |
| (mg/mL) | Testing | aration | T₀ | Tᵢₙᵢₜᵢₜₐₗ | hours | hours |
| 0.004 | Treprostinil | 1 | 97.8 | 94.8 | 96.7 | 99.9 |
| | Assay | 2 | 102.9 | 94.0 | 97.9 | 102.3 |
| | (% LC) | Average | 100.4 | 94.4 | 97.3 | 101.1 |
| | pH | NA | 6.4 | 6.6 | 6.6 | 6.6 |
| 0.13 | Treprostinil | 1 | 100.1 | 96.3 | 99.8 | 100.3 |
| | Assay | 2 | 100.5 | 96.0 | 99.7 | 100.2 |
| | (% LC) | Average | 100.3 | 96.1 | 99.7 | 100.2 |
| | pH | NA | 6.3 | 6.7 | 6.7 | 6.5 |

LC: Label claim

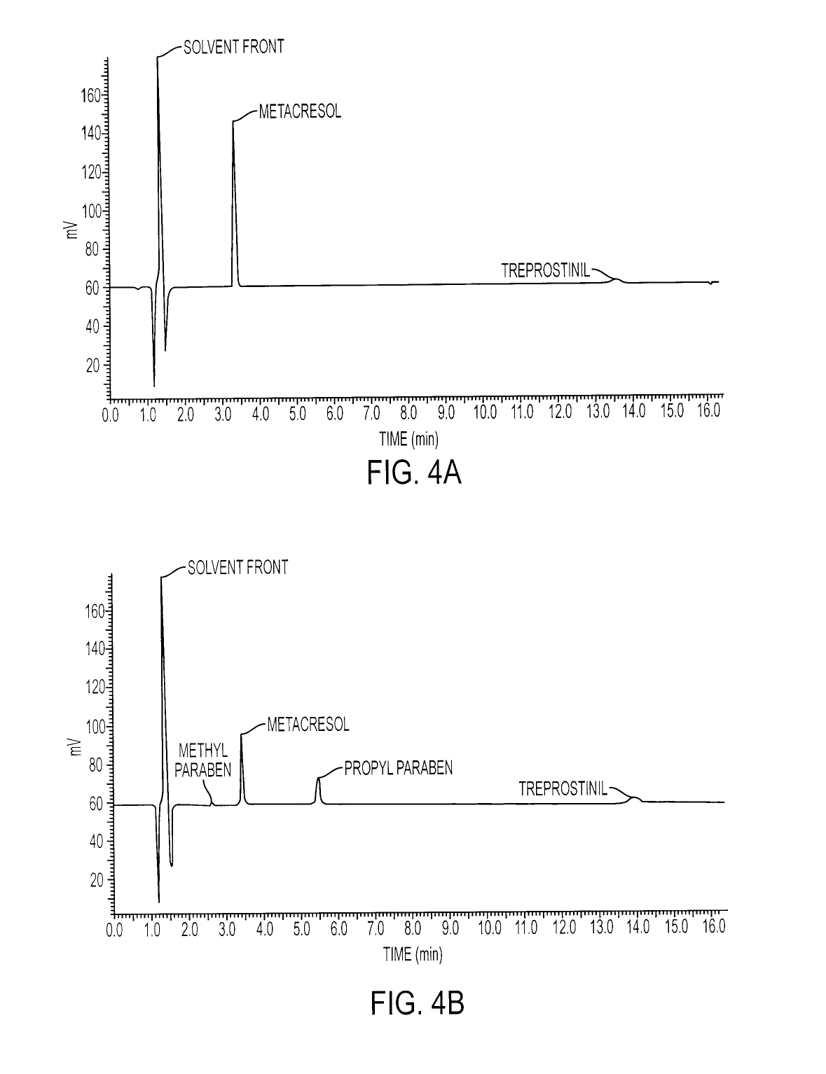

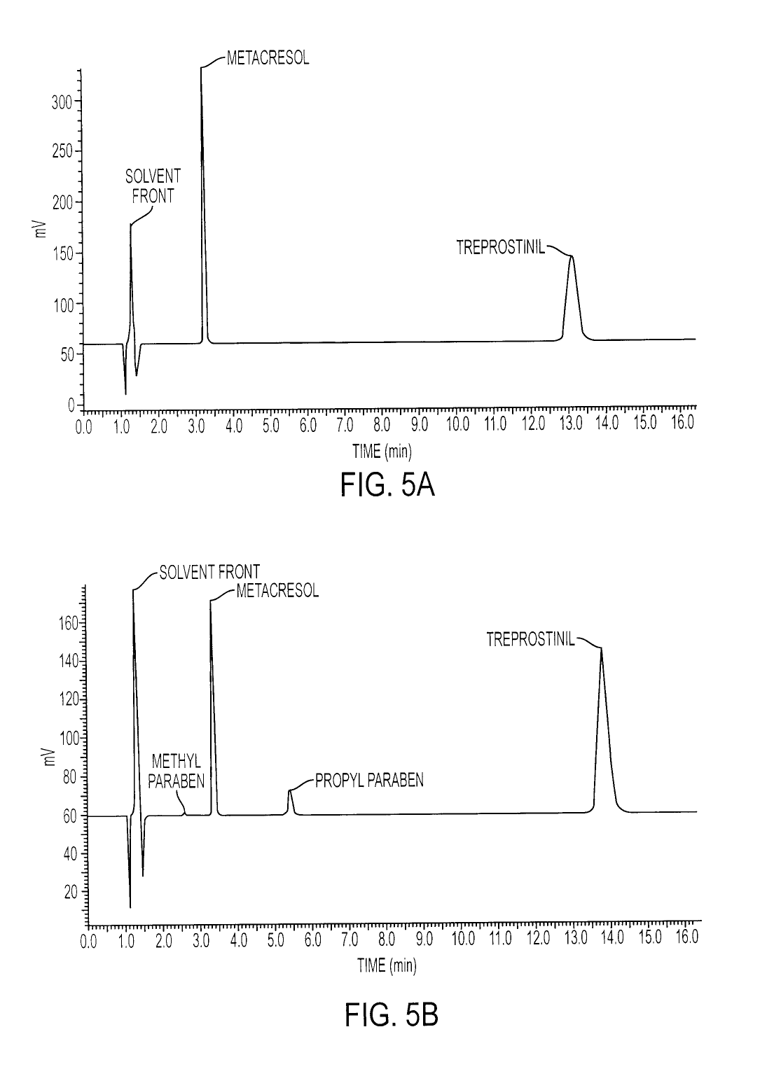

Similar results were obtained for the solutions of treprostinil in Sterile Diluent for FLOLAN®, which are summarized in Table 3. The appearance of all solutions was clear, colorless and free from visible particulate matter. The results also show no compatibility problems for the treprostinil solutions in Sterile Diluent for FLOLAN® for treprostinil at either concentration (FIGS. 4 and 5). Hence, the results show no compatibility problems for the dilute treprostinil solutions in any of the diluent solutions at either concentration.

TABLE 3
Chemical testing results for treprostinil in
Sterile Diluent for FLOLAN ®

| | | | Testing Interval | | | |
|---|---|---|---|---|---|---|
| Concentration | | Prep- | | | 24 | 52 |
| (mg/mL) | Testing | aration | T₀ | Tᵢₙᵢₜᵢₜₐₗ | hours | hours |
| 0.004 | Treprostinil | 1 | 95.9 | 108.4 | 100.6 | 100.7 |
| | Assay | 2 | 96.1 | 108.9 | 101.4 | 101.2 |
| | (% LC) | Average | 96.0 | 108.7 | 101.0 | 101.0 |
| | pH | NA | 10.6 | 10.5 | 10.6 | 10.5 |
| 0.13 | Treprostinil | 1 | 100.3 | 102.9 | 101.4 | 102.3 |
| | Assay | 2 | 100.2 | 102.7 | 101.4 | 102.1 |
| | (% LC) | Average | 100.2 | 102.8 | 101.4 | 102.2 |
| | pH | NA | 10.5 | 10.4 | 10.5 | 10.5 |

LC: Label claim

For the treprostinil solutions, after 52 hours in the cassette at 40° C./Ambient RH, the solutions were removed and AET was performed according to USP NF 24 Supplement 2<51> with an inclusion of a 48 hour plating for all organisms. For the FLOLAN® solution, the testing was performed following the same procedure, but after the solution had been in the cassette for 8 hours at room temperature. FLOLAN® was also tested for sterility.

The AET USP requirements for a Category 1 product, which includes parenteral solutions, are as follows: for bacteria, there must not be less than a 1.0 log reduction from the initial calculated count at 7 days and not less than a 3.0 log reduction from the initial count at 14 days and no increase from the 14 days' count at 28 days. For the yeast and mold, there should be no increase from the initial calculated count at 7, 14 and 28 days.

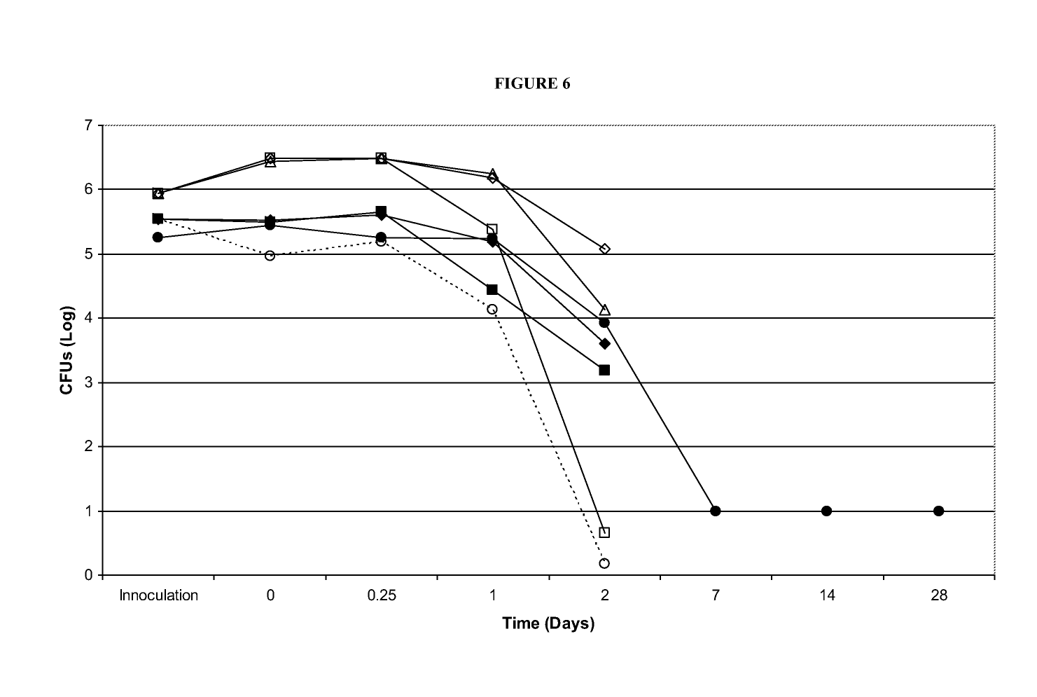

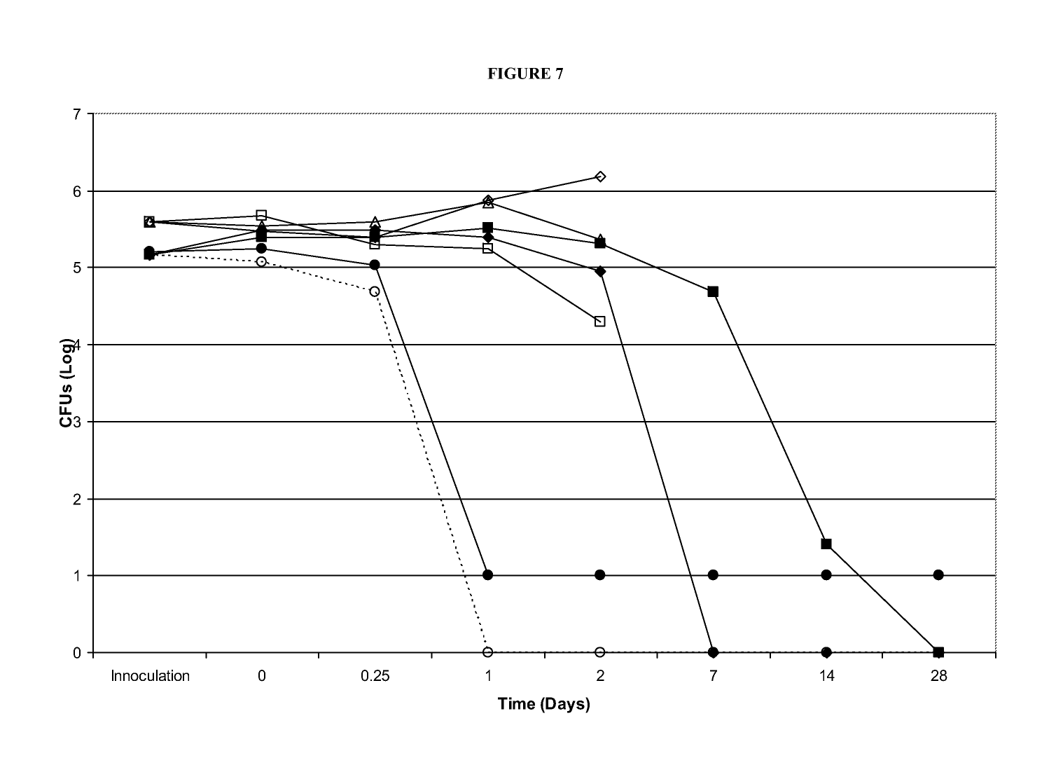

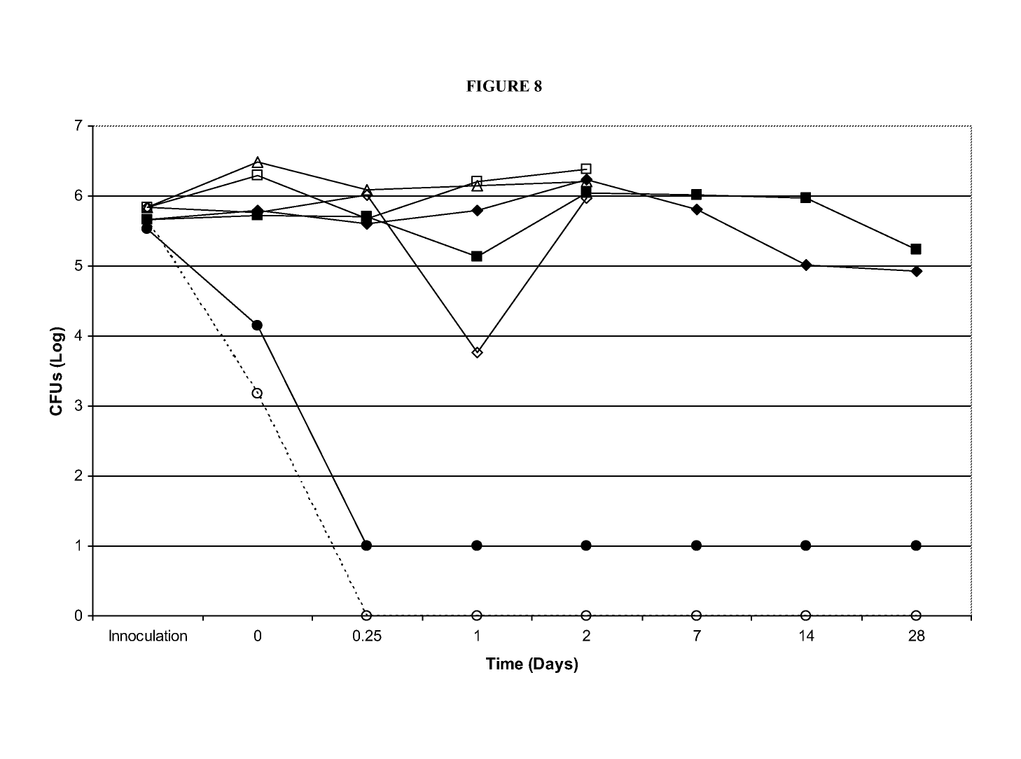

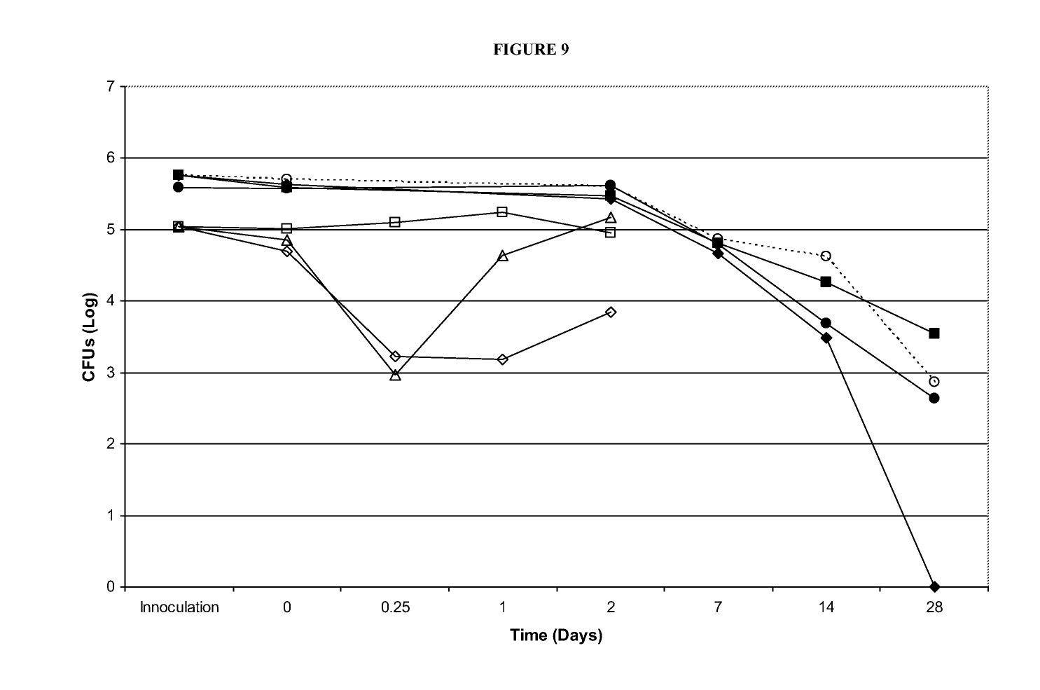

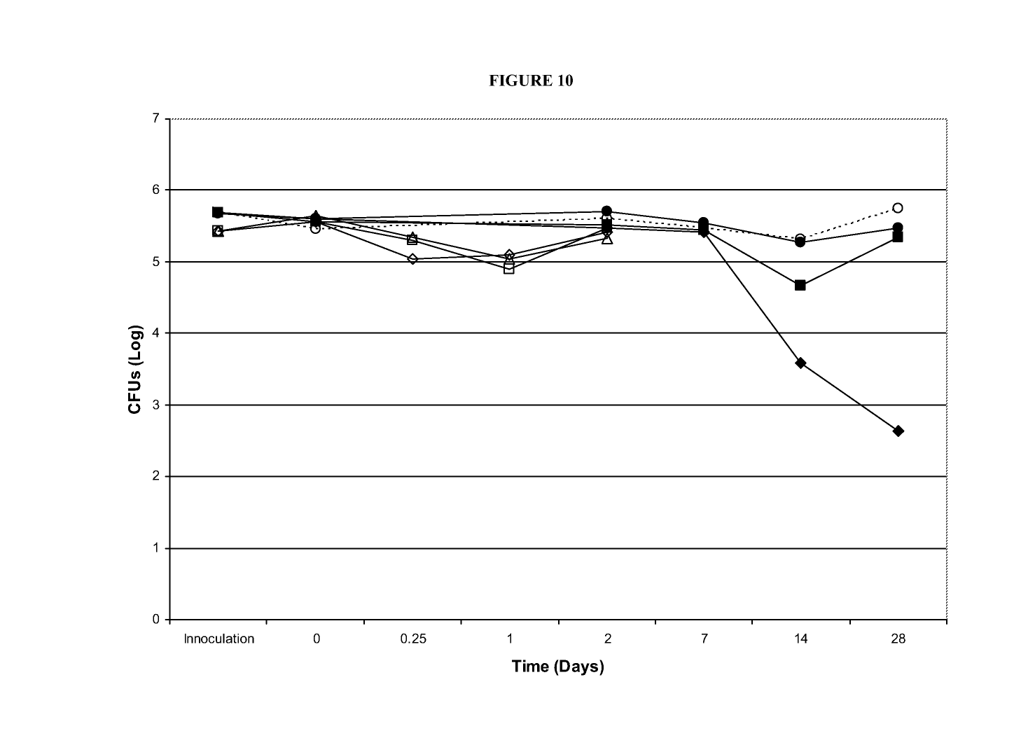

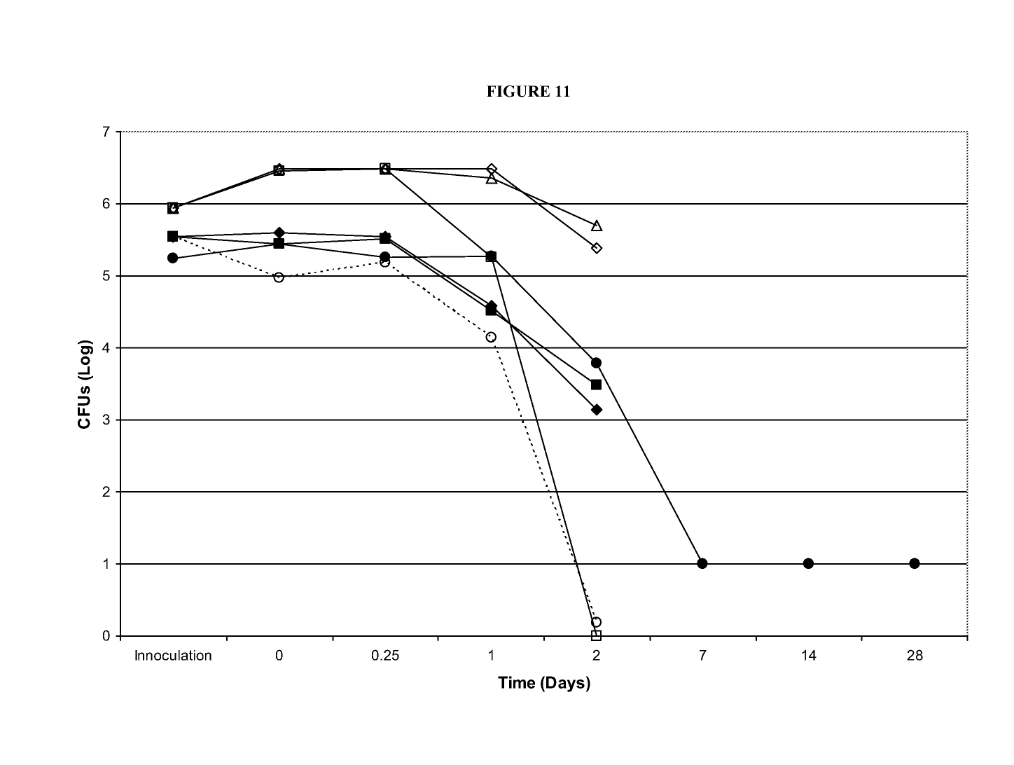

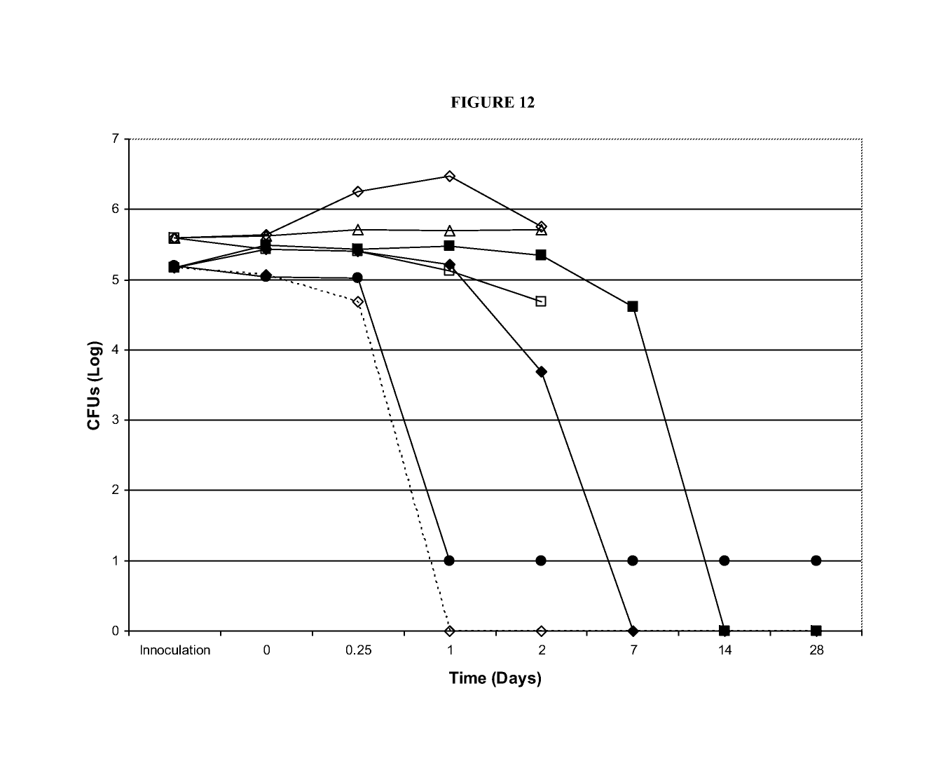

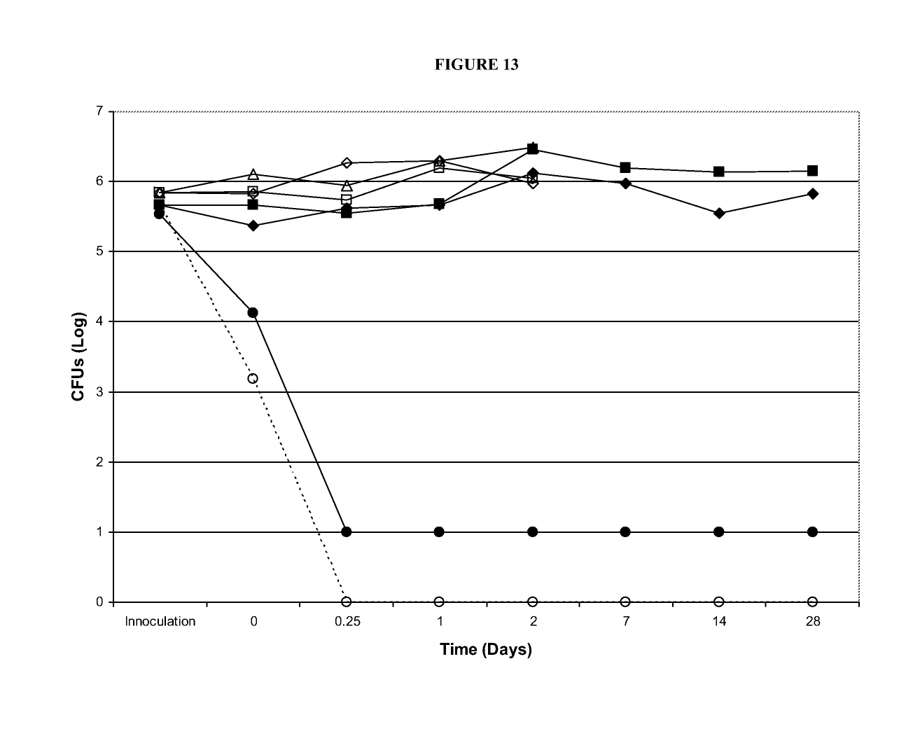

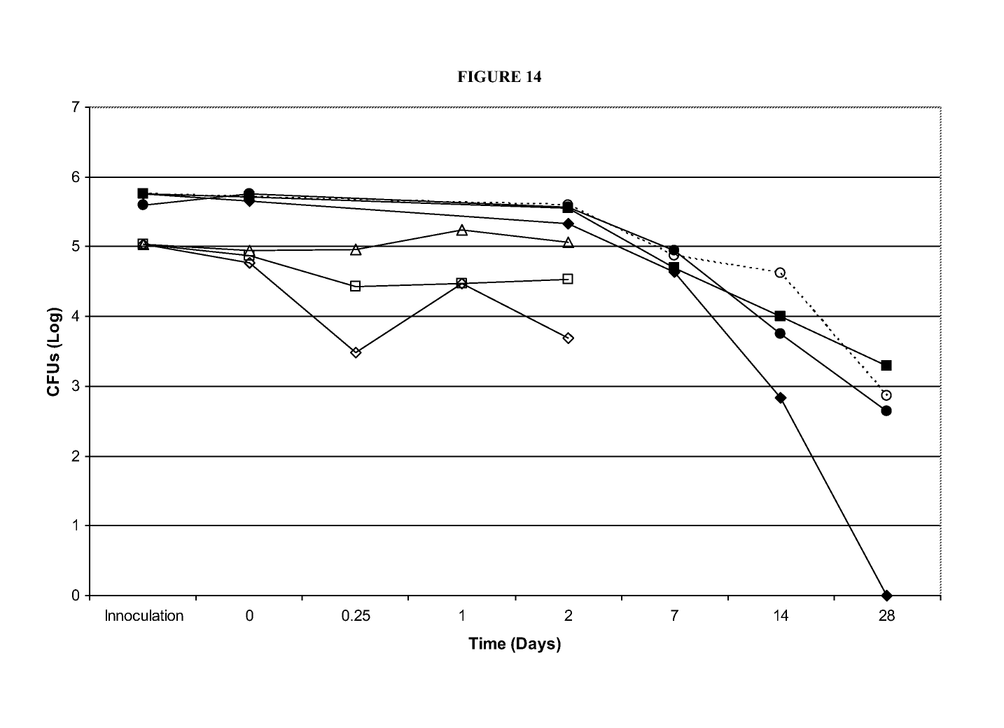

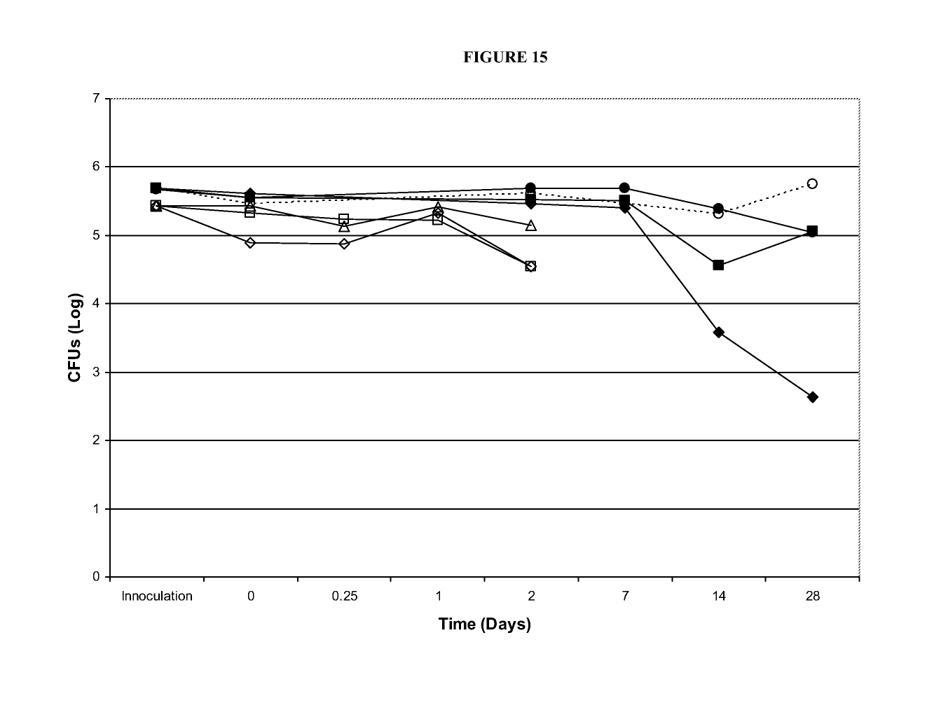

While FLOLAN® diluted in sterile diluent for Flolan met the USP requirements for AET, the treprostinil solutions in BWFI and BNS failed. These dilute treprostinil solutions failed AET because the bacterial reduction rate was not sufficient, mainly for gram negative bacteria. However, treprostinil in Sterile Diluent for FLOLAN® met the USP criteria. See FIGS. 6 to 15.

Three additional studies were performed that further support the use of dilute REMODULIN® for up to 19 days when Sterile Diluent for FLOLAN® (also referenced as “high pH Glycine diluent”) is used. One study evaluated the chemical stability of REMODULIN® diluted in Sterile Diluent for FLOLAN® for 19 days. A second study evaluated the ability of REMODULIN® diluted in Sterile Diluent for FLOLAN® to support microbial growth. A third study evaluated the ability of the inline 0.22-micron filter, included as part of the controlled ambulatory drug delivery (CADD) infusion system, to remove microbes from REMODULIN® diluted in normal saline.

The concentrations of treprostinil used for these studies were 0.004 and 0.130 mg/mL, as these treprostinil concentrations bracket the typical clinical concentrations (2.5 to 130 ng/kg/min, respectively) used by the majority of patients.

For the chemical stability study, the solutions were prepared by diluting REMODULIN® with a high pH glycine diluent, Sterile Diluent for FLOLAN®, to the targeted treprostinil concentrations of 0.004 and 0.130 mg/mL and stored at 5 and 25° C. in CADD cassette. For both concentrations and temperatures considered, the treprostinil concentrations remained constant within assay variability up to 19 days. Therefore, the chemical stability data supports that treprostinil is stable up to 19 days when diluted in a high-pH diluent and stored at either 5 or 25° C. in a CADD cassette.

The same treprostinil concentrations were prepared for the adventitious contamination 19-day study and were inoculated with microorganisms at levels of less than 500 CFU/mL. The inoculated solutions were stored in CADD cassettes at 2-8° C. or 20-25° C., and samples withdrawn from the cassettes at specific time intervals, up to 19 days, for enumeration of the test organism. The change in organism population over the study timeline was calculated as a log value. The bacteria (E. coli, S. aureus and P. aeruginosa) and yeast (C. albicans) showed population reduction over the duration of the study for each strength of product and each storage condition, and resulted in total population reduction by the last study time point. The mold (A. brasiliensis) showed no population increase over the duration of the study, nor did the organism population decrease over the duration of the study for both concentrations and each storage condition.

Per USP <51> no increase is defined as not more than 0.5 log increase. The data for the 19-day study shows that for all the microorganisms studied, the solutions did not support microbial growth at either 2-8° C. or 20-25° C.

A “Spike Recovery Study” was conducted to evaluate the efficiency of the 0.2-μm in-line filter to remove significant levels of microorganisms (yeast, mold, gram-positive cocci, gram-negative rods). REMODULIN® was diluted in normal saline to a treprostinil concentration of 0.130 mg/mL and spiked with high levels of each microorganism in a CADD cassette connected to a CADD pump and infusion set with an in-line 0.22-μm filter. The spiked microorganism levels exceeded those used in the adventitious contamination study described herein and those expected from contamination during normal preparation. Contaminated solutions were then pumped through the administration sets with samples collected at 4, 24, and 52 hours after pump initiation for microorganism enumeration. There was no recovery of any test organism from samples collected after the filter. Therefore, any inadvertent contamination of the final solution to be placed into a cassette would be removed by the robust in-line filter, which is at the distal end of the administration set, before coming into contact with a patient.

System suitability was performed per the method for each analysis set. All results met the method system suitability criteria. Chromatographic non-interference was demonstrated for each analysis set. See Table 4.1-1 for the results. These data support that the analytical method to assay treprostinil from Remodulin diluted with high-pH glycine diluent is acceptable.

TABLE 4.1-1
System Suitability Results (from 33 sets of analyses)

| | Result (Range) | Method Criteria |
|---|---|---|
| Initial precision (% RSD, n = 5) | 0.0-0.2 | ≦2.0 |
| Overall precision (% RSD) | 0.0-0.3 | ≦2.0 |
| Tailing factor | 1 | ≦2 |
| % Standard comparison | 99-101 | 98-102 |

RSD: relative standard deviation

The data generated for REMODULIN® diluted with high-pH glycine diluent to a treprostinil concentration of 0.004 mg/mL and stored at both conditions are shown in Table 4.1-2. The treprostinil levels remained constant throughout the 19-day study period, and changes noted are within assay variability. The target treprostinil concentration was 0.004 mg/mL. The percent label claim (% LC) values were normalized based on the initial assay value and labeled in the table as normalized percent label claim.

TABLE 4.1-2
Treprostinil (0.004 mg/mL) stability in High-pH Glycine Diluent at 5° C. and
25° C./60% RH followed by storage at ambient conditions

| | Time Stored at Ambient Conditions Following Pull (Hours) | | | | | | | |
|---|---|---|---|---|---|---|---|---|
| Pull | Hour 0/5° C. | | Hour 0/25° C. | | Hour 76/5° C. | | Hour 76/25° C. | |
| Time | | Norm | | Norm | | Norm | | Norm |
| (Day) | mg/mL | % LC | mg/mL | % LC | mg/mL | % LC | mg/mL | % LC |
| 0 | 0.00442 | 100.0 | 0.00442 | 100.0 | 0.00446 | 100.0 | 0.00446 | 100.0 |
| 1 | — | — | 0.00449 | 101.6 | — | — | 0.00448 | 100.4 |
| 2 | — | — | 0.00451 | 102.0 | — | — | 0.00447 | 100.2 |
| 3 | — | — | 0.00447 | 101.1 | — | — | 0.00448 | 100.4 |
| 4 | — | — | 0.00447 | 101.1 | — | — | 0.00444 | 99.6 |
| 9 | 0.00439 | 99.3 | 0.00454 | 102.7 | 0.00447 | 100.2 | 0.00452 | 101.3 |
| 11 | 0.00441 | 99.8 | 0.00452 | 102.3 | 0.00448 | 100.4 | 0.00452 | 101.3 |
| 15 | 0.00445 | 100.7 | 0.00451 | 102.0 | 0.00447 | 100.2 | 0.00454 | 101.8 |
| 19 | 0.00452 | 102.3 | 0.00455 | 102.9 | 0.00450 | 100.9 | 0.00450 | 100.9 |

Norm % LC is the normalized % label claim based on the initial value

The data generated for REMODULIN® diluted with high-pH glycine diluent to a treprostinil concentration of 0.130 mg/mL and stored at both conditions are shown in Table 4.1-3. The treprostinil levels remained consistent throughout the 19-day study period, and changes noted are within assay variability. The target treprostinil concentration was 0.130 mg/mL. The percent label claim (% LC) values were normalized based on the initial assay value and labeled in the table as normalized percent label claim.

TABLE 4.1-3
Treprostinil (0.130 mg/mL) stability in High-pH Glycine Diluent at 5° C. and
25° C./60% RH followed by storage at ambient conditions

| | Time Stored at Ambient Conditions Following Pull (Hours) | | | | | | | |
|---|---|---|---|---|---|---|---|---|
| Pull | Hour 0/5° C. | | Hour 0/25° C. | | Hour 76/5° C. | | Hour 76/25° C. | |
| Time | | Norm | | Norm | | Norm | | Norm |
| (Day) | mg/mL | % LC | mg/mL | % LC | mg/mL | % LC | mg/mL | % LC |
| 0 | 0.13622 | 100.0 | 0.13622 | 100.0 | 0.13650 | 100.0 | 0.13650 | 100.0 |
| 1 | — | — | 0.13601 | 99.8 | — | — | 0.13623 | 99.8 |
| 2 | — | — | 0.13791 | 101.2 | — | — | 0.13620 | 99.8 |
| 3 | — | — | 0.13640 | 100.1 | — | — | 0.13616 | 99.8 |
| 4 | — | — | 0.13759 | 101.0 | — | — | 0.13584 | 99.5 |
| 9 | 0.13510 | 99.2 | 0.13707 | 100.6 | 0.13616 | 99.8 | 0.13687 | 100.3 |
| 11 | 0.13572 | 99.6 | 0.13738 | 100.9 | 0.13687 | 100.3 | 0.13756 | 100.8 |
| 15 | 0.13652 | 100.2 | 0.13807 | 101.4 | 0.13662 | 100.1 | 0.13849 | 101.5 |
| 19 | 0.13800 | 101.3 | 0.14023 | 102.9 | 0.13658 | 100.1 | 0.13791 | 101.0 |

Norm % LC is the normalized % label claim based on the initial value

### 4.2 Adventitious Contamination Study

An adventitious contamination study was conducted to determine if REMODULIN® diluted with high-pH glycine diluent would support growth of specific microorganisms from potential contamination by a patient during dose preparation. The microorganisms selected for this study were based on the required organisms for an Antimicrobial Effectiveness Test (AET) as defined in the USP. REMODULIN® was diluted with high-pH glycine diluent and challenged with low levels of microorganisms as detailed above. As a control, normal saline was inoculated with these microorganisms and evaluated in the same manner. Tables 4.2-1 and 4.2-2 show the results from the contamination study for the treprostinil and control solutions, respectively.

TABLE 4.2-1
19-Day¹ Contamination Study of Remodulin in High-pH Glycine Diluent

| | CFU/mL (Time of Total Reduction if applicable) | | | | | |
|---|---|---|---|---|---|---|
| | | Treprostinil | | Treprostinil | | |
| | Intial | (0.004 mg/mL) | | (0.130 mg/mL) | | Log Reduction |
| | Inoculum | 2-8° C. | 20-25° C. | 2-8° C. | 20-25° C. | (2-8° C., 20-25° C.) |
| E. coli | 12 | 0 (H 52) | 0 (H 0) | 0 (H 26) | 0 (H 0) | >1.08 |
| S. aureus | 263 | 0 (H 76) | 0 (H 26) | 0 (H 52) | 0 (H 26) | >2.42 |
| P. aeruginosa | 59 | 0 (H 26) | 0 (H 26) | 0 (H 26) | 0 (H 26) | >1.77 |
| C. albicans | 131 | 1 | 0 (D 11) | 1 | 0 (D 12) | ≧2.12 |
| A. brasiliensis | 9.6 | 2 | 7 | 2 | 9 | 0.68, 0.13 |

H, Hour; D, Day
¹Data reported for the 19 days plus 76 hour time point, except where indicated

A total reduction in the E. coli, S. aureus and P. aeruginosa populations was observed as early as the first timepoint through the last timepoint evaluated for all conditions studied (Table 4.2-1). A total reduction of the C. albicans population was also observed at 20-25° C., and significantly reduced (to one CFU) at the last time point examined at 2-8° C. The A. brasiliensis population decreased over time at 2-8° C., and remained relatively constant over the duration of the study at 20-25° C.

TABLE 4.2-2
18-Day¹ Contamination Study of Saline Control

| | CFU/mL | | | |
|---|---|---|---|---|
| | Initial | Saline | | Log Reduction |
| | Inoculum | 2-8° C. | 20-25° C. | (2-8° C., 20-25° C.) |
| E. coli | 12 | 0 | >300 | >1.08, >−1.40 |
| S. aureus | 263 | 0 | 0 | >2.42 |
| P. aeruginosa | 59 | 89 | >300 | −0.18, >−0.71 |
| C. albicans | 131 | 8 | 0 | 1.22, >2.12 |
| A. brasiliensis | 9.6 | 8 | 15 | 0.08, −0.20 |

¹Data reported for the 18 days plus 72 hour time point

In saline control solutions, the E. coli, S. aureus, C. albicans and A. brasiliensis populations decreased over the study period and the P. aeruginosa population remained constant over the duration of the study with a slight increase observed at the final time point at 2-8° C. At 20-25° C., the S. aureus, C. albicans and A. brasiliensis populations decreased over the study period, whereas the E. coli and P. aeruginosa populations increased to an uncountable range during the study. The Saline Control results are for informational purposes only.

### 4.3 Spike Recovery Studies for Dilute Remodulin® Solutions

The data generated from the spike recovery study is presented in Table 4.3-1. The inoculum level for each organism represents a challenge equivalent to the antimicrobial effectiveness test and represents a level significantly greater than expected from inadvertent contamination resulting from labeled use.

TABLE 4.3-1
Microorganism levels after the in-line filter

| | | Effluent | | | Positive Control |
|---|---|---|---|---|---|
| | | Bioburden (CFU) | | | (Cassette Sample) |
| | Inoculum | Hour | Hour | Hour | (CFU/mL) |
| Organism | Level (CFU) | 4 | 24 | 52 | Hour 52 |
| S. aureus | 5.75 × 10⁵ | 0 | 0 | 0 | 4 |
| P. aeruginosa | 9.85 × 10⁵ | 0 | 0 | 0 | >300 |
| E. coli | 5.05 × 10⁵ | 0 | 0 | 1¹ | >300 |
| C. albicans | 6.45 × 10⁵ | 0 | 0 | 0 | >300 |
| A. niger | 2.40 × 10⁶ | 0 | 0 | 0 | >300 |

¹Atypical growth observed (1 -yellow, round, flat). A Gram stain was performed following ATM-MBU-M0014.04 with a result of “Gram Positive Rods,” therefore, the organism was a contaminant and not E. coli.

There was no recovery of any of the test organisms in the effluent at 4, 24, or 52 hours. At 52 hours, one CFU was recovered in the sample collected in the effluent for the sample spiked with E. coli. This organism was shown to be a gram-positive rod by gram stain. The spiked organism, E. coli, is a gram-negative rod and therefore the growth observed is likely a laboratory contaminant introduced during sample collection or in processing the sample. Gram-positive rods are commonly found in the environment of the laboratory. At the completion of effluent collection, viability of the inoculum was confirmed by plating some of the remaining spiked sample.

Taken together, the chemical stability and microbiological data further support use of REMODULIN® diluted with a high-pH glycine diluent up to 19 days. Furthermore, the spike recovery study demonstrates that the 0.2-μm in-line filter is efficient at removing any potential contaminants that may be introduced upon dose preparation.

While the invention has been described in connection with specific embodiments thereof, it will be understood that it is capable of further modifications and this application is intended to cover any variations, uses, or alterations of the invention following. In general, the principles of the invention and including such departures from the present disclosure as come within known or customary practice within the art to which the invention pertains and as may be applied to the essential features hereinbefore set forth and as follows in the scope of the appended claims.

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention.

What is claimed is:

1. A method of treating pulmonary hypertension, comprising combining treprostinil or treprostinil sodium with a diluent providing a pH of about 10 or greater to form a diluted solution of treprostinil, maintaining the diluted solution of treprostinil for a period of up to 14 days prior to administration, and starting to administer the diluted solution of treprostinil to a patient suffering from pulmonary hypertension within a period of up to 14 days following dilution.

2. The method of claim 1, wherein the diluted solution of treprostinil is administered by intravenous injection.

3. The method of claim 1, wherein the diluent further comprises glycine.

4. The method of claim 3, wherein the pH is between 10.2 and 10.8

5. The method of claim 4, wherein the diluted solution of treprostinil is administered by intravenous injection.

6. The method of claim 1, wherein the diluted solution of treprostinil further comprises arginine.
